(19) United States
(12) Patent Application Publication
(10) Pub. No.: US 2006/0287219 A1
(43) Pub. Date: Dec. 21, 2006

(54) Benefit agent delivery systems

(76) Inventors: Robert Richard Dykstra, Cleves, OH (US); Lon Montgomery Gray, Florence, KY (US); Lois Sara Gallon, Finneytown, OH (US); Johan Smets, Lubbeek (BE); Abdennaceur Fredj, Loveland, OH (US); Daniel Jerome White JR., West Chester, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/513,633

(22) Filed: Aug. 31, 2006

Related U.S. Application Data
(62) Division of application No. 10/269,274, filed on Oct. 11, 2002.
(60) Provisional application No. 60/359,644, filed on Oct. 19, 2001.

(51) Int. Cl. A61K 8/18

(52) U.S. Cl. 512/10

(57) ABSTRACT

Disclosed herein are benefit agent delivery systems which are formed by separately adding to a liquid or granular matrix certain kinds of primary amine compounds and selected types of benefit agents, e.g., perfumes, in the form of aldehydes or ketones. When substrate surfaces are treated with aqueous solutions or dispersions of such delivery systems, the benefit agent is indirectly exposed to and preferably deposited on the substrate surface in such a manner that it provides its benefit to the surface for a longer period of time than when the amine compound is not present. Such benefit agent delivery systems are especially suitable for incorporation into laundry detergent or other fabric-treating products.

### CROSS REFERENCE

This application is a divisional of and claims priority under 35 U.S.C. §120 to U.S. application Ser. No. 10/269,274, filed Oct. 11, 2002, which in turn claims benefit of U.S. Application Ser. No. 60/359,644, filed Oct. 19, 2001.

### TECHNICAL FIELD

The present invention relates to benefit agent delivery systems which can be used to deposit benefit agents such as perfumes onto the surface of a substrate, e.g., fabrics being laundered, hard surfaces, hair, or skin.

### BACKGROUND OF THE INVENTION

It is frequently desirable or advantageous to treat the surfaces of a variety of substrates, for example fabrics, skin, hair, etc., with benefit agents such as perfumes, flavors, pharmaceuticals and/or biocontrol agents including biocides, insecticides, mildewcides, and the like. The objective of such treatment is generally to leave deposited on the surfaces of the substrates enough benefit agent so that there is a residual benefit imparted to the substrate surface after treatment of the substrate is completed.

Products, systems and methods for depositing benefit agents onto the surfaces of substrates are well known in the art. For example, in the context of fabric treatment such as fabric laundering, a variety of laundry and other products exist which can be used to form aqueous washing liquors or rinse baths containing benefit agents which deposit onto the surfaces of fabrics which are contacted with such liquors or baths.

One type of laundry product which involves the improved deposition of perfume materials onto fabrics laundered with such products is described in U.S. Pat. No. 6,103,678. This '678 patent discloses laundry detergent or other treatment compositions which utilize the combination of an amino-functional polymer and a selected type of hydrophilic perfume in order to obtain effective perfume substantivity on fabrics laundered using such compositions.

Other types of products which provide improved deposition onto substrate surfaces of benefit agents such as perfumes are described in PCT Patent Application Nos. WO 00/02991; WO 00/02981; WO 00/02987 and WO 00/02982. These patent publications disclose compositions wherein benefit agent substantivity on treated substrates is realized by incorporating into substrate treatment products a reaction product formed from amine-based compounds and certain types of benefit agents which are pre-reacted with such amine-based compounds.

However, notwithstanding the advances in the art as represented by the foregoing patent and patent publications, there remains a continuing need to identify benefit agent delivery systems which are especially effective for delivering residual and long-lasting benefit agents to substrates treated using such delivery systems.

### SUMMARY OF THE INVENTION

The present invention provides a benefit agent delivery system suitable for delivering a benefit agent to the surface of a substrate. Such a delivery system comprises a liquid or granular matrix to which is separately added both an amine-based compound and a benefit agent in the form of an aldehyde or ketone. The amine-based compound is preferably a polyamine and will have a molecular weight greater than 100 Daltons. At least 10% of the amino groups of the amine-based compound must be primary amino groups.

The benefit agent and the separately added amine-based compound are selected and formulated together so that they will be exposed to and preferably deposit onto the surface of the substrate by means of contact of the substrate surface with a diluted, and preferably an aqueous, solution or dispersion of the delivery system. When this happens, the benefit agent will provide its benefit on and to the surface of the substrate for a longer period of time than when the amine-based compound is not present.

Most preferably the delivery system will comprise a liquid or granular laundry detergent or fabric-treating composition. Most preferably also the benefit agent which is delivered will be a perfume material. Compositions in the form of body wash products and shampoos are also contemplated.

### DETAILED DESCRIPTION OF THE INVENTION

The essential components of the benefit agent delivery systems of the present invention include a liquid or granular matrix to which is separately added an amine compound and a benefit agent in certain chemical form. Each of these components is discussed in detail as follows along with other elements of the delivery systems herein as well as methods for their preparation and use.

Liquid or Granular Matrix

The benefit agent delivery systems herein are based on the formation of a liquid or granular matrix. “Liquids” include fluids of density and viscosity which are conventional for liquids as well as gels and foams. Useful liquids may be aqueous or non-aqueous. Water is typically the major component of the delivery systems which are in aqueous liquid form. Conventional non-aqueous solvents may be used to form the matrix for liquid delivery systems in non-aqueous form. Liquid products, i.e., those containing 10% or greater of water or other solvents, are highly preferred.

Delivery systems in granular form can be fashioned from any type of solid-state material which comprises particles or granules ranging in size from 1 μm to 100 mm. Thus the granular matrix can include particles ranging from very fine powder to agglomerates or tablets. The granular matrix furthermore can comprise either inert or active ingredients, or both, with respect to the function of the product into which the delivery system is to be incorporated.

Most typically, the liquid or granular matrix used to form the delivery systems herein will comprise the matrix for the liquid or granular end product into which the benefit agent delivery system will be incorporated and made a part of. Thus, for example, liquid or granular detergent compositions for laundry or hard surface cleaning will frequently comprise the liquid or granular matrix into which the amine-based compounds and benefit agents described herein will be separately added to form the delivery systems of this invention.

Separate Addition of Amine Compound and Benefit Agent

It is an essential feature of the present invention that the amine compound and the benefit agent be added separately to the liquid or granular matrix. For purposes of this invention, the amine-based compound and benefit agent are separately added to the system-forming matrix if the entire amounts of these components are combined with the matrix as discrete components. In particular, there must be essentially no chemical reaction between these two materials before they are combined with the matrix. Thus the amine compound and the benefit agent may be added to the matrix at separate times and/or from separate containers or from separate holding or delivery means. The amine compound and the benefit agent materials may even be mixed together prior to combination with the system-forming matrix so long as substantially no chemical reaction occurs between these materials prior to their contact with the system-forming matrix.

Amine-Based Compound

The amine-based compound which is added to the liquid or granular matrix as part of delivery system preparation may be a mono-amine or a polyamine so long as its molecular weight is greater than 100 Daltons and so long as at least 10% of its amino groups are primary amino groups. Preferably the amino-based compound will be a polyamine, the molecular weight of the compound will be at least 150 Daltons, and from 15% to 80% of its amino groups will be primary amino groups.

The amine-based compounds used in this invention are also preferably ones characterized by having an Odor Intensity Index of less than that of a 1% solution of methylanthranilate in dipropylene glycol.

Odor Intensity Index Method

Odor Intensity Index is a value determined by expert graders who evaluate test chemicals for odor when such the pure chemicals are diluted at 1% in dipropylene glycol (DPG), odor-free solvent used in perfumery. This concentration percentage is representative of typical usage levels. Smelling strips, or so called “blotters”, were are dipped in test solutions and presented to the expert panelist for evaluation. Expert panelists are assessors trained for at least six months in odor grading and whose grading are checked for accuracy and reproducibility versus a reference on an on-going basis. For each amine compound, a panelist is presented two blotters: one reference (Me Anthranilate, unknown from the panelist) and the test sample. The panelist is asked to rank both smelling strips on the 0-5 odor intensity scale, 0 being no odor detected, 5 being very strong odor present.

Results:

The following represents Odor Intensity Index of some amine compounds suitable for use in the present invention and according to the above procedure. In each case, numbers are arithmetic averages among 5 expert panelists and the results are statistically significantly different at 95% confidence level:

| | |
|---|---|
| Methylanthranilate 1% (reference) | 3.4 |
| Ethyl-4-aminobenzoate (EAB) 1% | 0.9 |
| 1,4-bis-(3-aminopropyl)-piperazine (BNPP) 1% | 1.0 |

A wide variety of primary amine-based compounds which have the preferred Odor Intensity Index characteristics can be used to prepare the benefit agent delivery systems of this invention. A general structure for a primary amine compound useful in this invention is as follows:
B—(NH₂)ₙ;
wherein B is a carrier material, and n is an index of value of at least 1. Compounds containing a secondary amine group have a structure similar to the above with the exception that the compound comprises one or more —NH— groups as well as —NH₂ groups. Preferably the amine compounds of this general type will be relatively viscous materials. Suitable B carriers include both inorganic and organic carrier moieties. By “inorganic carrier”, it is meant a carrier which is comprised of non- or substantially non-carbon based backbones.

Preferred primary amines, utilizing inorganic carriers, are those selected from mono or polymers or organic-organosilicon copolymers of amino derivatised organo silane, siloxane, silazane, alumane, aluminum siloxane, or aluminum silicate compounds. Typical examples of such carriers are: organosiloxanes with at least one primary amine moiety like the diaminoalkylsiloxane [H₂NCH₂(CH₃) 2Si]O, or the organoaminosilane (C₆H₅) 3SiNH₂ described in: Chemistry and Technology of Silicone, W. Noll, Academic Press Inc. 1998, London, pp 209, 106).

Preferred primary amines, utilizing organic carriers, are those selected from aminoaryl derivatives, polyamines, amino acids and derivatives thereof, substituted amines and amides, glucamines, dendrimers, polyvinylamines and derivatives thereof, and/or copolymer thereof, alkylene polyamine, polyaminoacid and copolymer thereof, cross-linked polyaminoacids, amino substituted polyvinylalcohol, polyoxyethylene bis amine or bis aminoalkyl, aminoalkyl piperazine and derivatives thereof, bis(amino alkyl)alkyl diamine linear or branched, and mixtures thereof.

Preferred aminoaryl derivatives are the amino-benzene derivatives including the alkyl esters of 4-amino benzoate compounds, and more preferably selected from ethyl-4-amino benzoate, phenylethyl-4-aminobenzoate, phenyl-4-aminobenzoate, 4-amino-N′-(3-aminopropyl)-benzamide, and mixtures thereof.

Polyamines suitable for use in the present invention are polyethyleneimine polymers, partially alkylated polyethylene polymers, polyethyleneimine polymers with hydroxyl groups, 1,5-pentanediamine, 1,6-hexanediamine, 1,3 pentanediamine, 3-dimethylpropanediamine, 1,2-cyclohexanediamine, 1,3-bis(aminomethyl)cyclohexane, tripropylenetetraamine, bis(3-aminopropyl)piperazine, dipropylenetriamine, tris(2-aminoethylamine), tetraethylenepentamine, bishexamethylenetriamine, bis(3-aminopropyl) 1,6-hexamethylenediamine, 3,3′-diamino-N-methyldipropylamine, 2-methyl-1,5-pentanediamine, N,N,N′,N′-tetra(2-aminoethyl)ethylenediamine, N,N,N′,N′-tetra(3-aminopropyl)-1,4-butanediamine, pentaethylhexamine, 1,3-diamino-2-propyl-tert-butylether, isophorondiamine, 4,4′,-diaminodicyclohylmethane, N-methyl-N-(3-aminopropyl)ethanolamine, spermine, spermidine, 1-piperazineethaneamine, 2-(bis(2-aminoethyl)amino)ethanol, ethoxylated N-(tallowalkyl)trimethylene diamines,poly[oxy(methyl-1,2-ethanediyl)], α-(2-aminomethyl-ethoxy)-(=C.A.S No. 9046-10-0); poly[oxy(methyl-1,2-ethanediyl)], α-hydro-)-ω-(2-aminomethylethoxy)-, ether with 2-ethyl-2-(hydroxymethyl)-1,3-propanediol (=C.A.S. No. 39423-51-3); commercially available under the tradename Jeffamines T-403, D-230, D-400, D-2000; 2,2′,2″-triaminotriethylamine; 2,2′-diamino-diethylamine; 3,3′-diamino-dipropylamine, 1,3 bis aminoethyl-cyclohexane commercially available from Mitsubishi and the C12 Sternamines commercially available from Clariant like the C12 Sternamin(propylenamine)ₙ with n=¾, and mixtures thereof. Preferred polyamines are polyethyleneimines commercially available under the tradename Lupasol like Lupasol FG (MW 800), G20wfv (MW 1300), PR8515 (MW 2000), WF (MW 25000), FC (MW 800), G20 (MW 1300), G35 (MW 1200), G100 (MW 2000), HF (MW 25000), P (MW 750000), PS (MW 750000), SK (MW 2000000), SNA (MW 1000000). Of these, the most preferred include Lupasol HF or WF (MW 25000), P (MW 750000), PS (MW 750000), SK (MW 2000000), 620wfv (MW 1300) and PR 1815 (MW 2000), Epomin SP-103, Epomin SP-110, Epomin SP-003, Epomin SP-006, Epomin SP-012, Epomin SP-018, Epomin SP-200, and partially alkoxylated polyethyleneimine, like polyethyleneimine 80% ethoxylated from Aldrich.

Preferred amino acids for use herein are selected from tyrosine, tryptophane, lysine, glutamic acid, glutamine, aspartic acid, arginine, asparagine, phenylalanine, proline, serine, histidine, threonine, methionine, and mixture thereof, most preferably selected from tyrosine, tryptophane, and mixture thereof. Preferred amino acid derivatives are selected from tyrosine ethylate, glycine methylate, tryptophane ethylate, and mixture thereof.

Preferred substituted amines and amides for use herein are selected from nipecotamide, N-coco-1,3-propenediamine; N-oleyl-1,3-propenediamine; N-(tallow alkyl)-1,3-propenediamine; 1,4-diamino cyclohexane; 1,2-diamino-cyclohexane; 1,12-diaminododecane, and mixtures thereof.

Other primary amine compounds suitable for use herein are the glucamines, preferably selected from 2,3,4,5,6-pentamethoxy-glucamine; 6-acetylglucamine, glucamine, and mixture thereof.

Also preferred compounds are the polyethylenimine and/or polypropylenimine dendrimers and the commercially available Starburst® polyamidoamines (PAMAM) dendrimers, generation G0-G10 from Dendritech and the dendrimers Astromols®, generation 1-5 from DSM being DiAminoButane PolyAmine DAB (PA)x dendrimers with x=2ⁿ×4 and n being generally comprised between 0 and 4.

Polyamino acid is one suitable class of amino-based compound useful herein. Polyaminoacids are compounds which are made up of amino acids or chemically modified amino acids. They can contain alanine, serine, aspartic acid, arginine, valine, threonine, glutamic acid, leucine, cysteine, histidine, lysine, isoleucine, tyrosine, asparagine, methionine, proline, tryptophan, phenylalanine, glutamine, glycine or mixtures thereof. In chemically modified amino acids, the amine or acidic function of the amino acid has reacted with a chemical reagent. This is often done to protect these chemical amine and acid functions of the amino acid in a subsequent reaction or to give special properties to the amino acids, like improved solubility. Examples of such chemical modifications are benzyloxycarbonyl, aminobutyric acid, butyl ester, pyroglutamic acid. More examples of common modifications of amino acids and small amino acid fragments can be found in the Bachem, 1996, Peptides and Biochemicals Catalog.

A preferred polyamino acid is polylysine. Most preferred are polylysines or polyamino acids where more than 50% of the amino acids are lysine, since the primary amine function in the side chain of the lysine is the most reactive amine of all amino acids.

The preferred polyamino acid has a molecular weight of 500 to 10,000,000, more preferably between 2000 and 25,000.

The polyamino acid can be cross linked. The cross linking can be obtained for example by condensation of the amine group in the side chain of the amino acid like lysine with the carboxyl function on the amino acid or with protein cross linkers like PEG derivatives. The cross linked polyamino acids still need to have free primary and/or secondary amino groups left for reaction with the active ingredient.

The preferred cross linked polyamino acid has a molecular weight of 20,000 to 10,000,000; more preferably between 200,000 and 2,000,000.

The polyamino acid or the amino acid can be co-polymerized with other reagents like for instance with acids, amides, acyl chlorides. More specifically with aminocaproic acid, adipic acid, ethylhexanoic acid, caprolactam or mixture thereof. The molar ratio used in these copolymers ranges from 1:1 (reagent/amino acid (lysine)) to 1:20, more preferably from 1:1 to 1:10.

The polyamino acid like polylysine can also be partially ethoxylated so long as the requisite amount of primary amino groups remains in the polymer. Preferably, however, the amine-based compounds utilized herein are unethoxylated.

Examples and supply of polyaminoacids containing lysine, arginine, glutamine, asparagine are given in the Bachem 1996, Peptides and Biochemicals catalog.

The polyaminoacid can be obtained before reaction with the active ingredient, under a salt form. For example polylysine can be supplied as polylysine hydrobromide. Polylysine hydrobromide is commercially available from Sigma, Applichem, Bachem and Fluka.

Examples of suitable amino functional polymers containing at least one primary amine group for the purposes of the present invention are:

Polyvinylamine with a MW of 300-2.10E6;

Polyvinylamine alkoxylated with a MW of 600, 1200 or 3000 and an ethoxylation degree of 0.5;

Polyvinylamine vinylalcohol—molar ratio 2:1, polyvinylaminevinylformamide—molar ratio 1:2 and polyvinylamine vinylformamide-molar ratio 2:1;

Triethylenetetramine, diethylenetriamine, tetraethylenepentamine;

Bis-aminopropylpiperazine;

Polyamino acid (L-lysine/lauric acid in a molar ratio of 10/1), Polyamino acid (L-lysine/aminocaproic acid/adipic acid in a molar ratio of 5/5/1), ), Polyamino acid (L-lysine/aminocaproic acid/ethylhexanoic acid in a molar ratio of 5/3/1) Polyamino acid (polylysine-cocaprolactam); Polylysine; Polylysine hydrobromide; cross-linked polylysine,

amino substituted polyvinylalcohol with a MW ranging from 400-300,000;

polyoxyethylene bis [amine] available from e.g. Sigma;

polyoxyethylene bis [6-aminohexyl] available from e.g. Sigma;

N,N′-bis-(3-aminopropyl)-1,3-propanediamine linear or branched (TPTA);

N,N′-bis-(3-aminopropyl)ethylenediamine; and

-1,4-bis-(3-aminopropyl)piperazine (BNPP).

The most preferred amine compounds for use herein will be non-aromatic amines. These most preferred amine compounds are selected from polyethyleneimine polymers commercially available under the tradename Lupasol like Lupasol HF, P, PS, SK, SNA, WF, G20wfv and PR8515; the diaminobutane dendrimers Astramol®, polylysine, cross-linked polylysine, N,N′-bis-(3-aminopropyl)-1,3-propanediamine linear or branched; N,N′-bis-(3-aminopropyl)-ethylenediamine; 1,4-bis-(3-aminopropyl)piperazine, and mixtures thereof. Even more preferred compounds are those selected from polyethyleneimine polymers having a molecular weight grater than 200 daltons including those commercially available under the tradename Lupasol like Lupasol HF, P, PS, SK, SNA, WF, G20wfv and PR8515; polylysine, cross-linked polylysine, N,N′-bis-(3-aminopropyl)-1,3-propanediamine linear or branched, N,N′-bis-(3-aminopropyl)-ethylenediamine; 1,4-bis-(3-aminopropyl)piperazine, and mixtures thereof.

As noted, the amine component of the delivery systems herein may also be a monoamine. Nonlimiting examples of suitable monoamines for use in the present invention include, but are not limited to, primary amines that also contain hydroxy and/or alkoxy functional groups, such as the 2-hydroxyamines and/or 3-hydroxyamines, primary and/or secondary amines that also contain a functional group that enhances deposition of the monoamine compared to monoamines that lack that functional group, especially when the monoamine is interacting with the benefit agent.

When the amine is a monoamine, it is preferred that the monoamine have certain solubility characteristics as measured by ClogP. The ClogP value is a measurement of the octanol/water partition coefficient of the monoamine molecule and is the ratio between its equilibrium concentrations in octanol and in water. Since the partition coefficients of the monoamine materials useful herein have high values, they are more conveniently given in the form of their logarithm to the base 10, logP, which is known as the ClogP value. ClogP is defined in the following references: “Calculating log P_{oct} from Structures”; Albert Leo (Medicinal Chemistry Project, Pomona College, Claremont, Calif. USA. Chemical Reviews, Vol. 93, number 4, June 1993; as well as from Comprehensive Medicinal Chemistry, Albert Leo, C. Hansch, Ed. Pergamon Press: Oxford, 1990, Vol. 4, p. 315; and Calculation Procedures for molecular lipophilicity: a comparative Study, Quant. Struct. Act. Realt. 15, 403-409 (1996), Raymund Mannhold and Karl Dross. The preferred monoamines for use herein are those having a ClogP greater than 1, preferably greater than 2.

Primary monoamines may also be used herein in combination with secondary monoamines. However, enough of the primary monoamine must be used to provide at least 10% of the total amine groups within such combinations as primary amine groups.

Benefit Agent

Another essential component of the benefit agent delivery systems herein is a benefit agent itself. The benefit agents essentially used to form the delivery systems of this invention must be in the form of an aldehyde or ketone.

The benefit agent can, for example, be selected from a flavor ketone or aldehyde, a pharmaceutical ketone or aldehyde, a biocontrol ketone or aldehyde, a perfume ketone or aldehyde and mixtures thereof.

Flavor ingredients include spices or flavor enhancers which contribute to the overall flavor perception of the product into which the benefit agent delivery system is incorporated. Pharmaceutical benefit agents include drugs. Biocontrol agents include biocides, antimicrobials, bactericides, fungicides, algaecides, mildewcides, disinfectants, sanitizer-like bleaches, antiseptics, insecticides, insect and/or moth repellant, vermicides, plant growth hormones, and the like.

Typical antimicrobials include glutaraldehyde, cinnamaldehyde, and mixtures thereof. Typical insect and/or moth repellants are perfume ingredients, such as citronellal, citral, N,N diethyl meta toluamide, Rotundial, 8-acetoxycarvotanacenone, and mixtures thereof. Other examples of insect and/or moth repellant for use as benefit agents herein are disclosed in U.S. Pat. Nos. 4,449,987, 4,693,890, 4,696,676, 4,933,371, 5,030,660, 5,196,200, and “Semio Activity of Flavor and Fragrance molecules on various Insect Species”, B. D. Mookherjee et al., published in Bioactive Volatile Compounds from Plants, ASC Symposium Series 525, R. Teranishi, R. G. Buttery, and H. Sugisawa, 1993, pp. 35-48.

A typical disclosure of suitable ketone and/or aldehydes, traditionally used in perfumery, can be found in “Perfume and Flavor Chemicals”, Vol. I and II, S. Arctander, Allured Publishing, 1994, ISBN 0-931710-35-5. Pefume ketones and aldehydes are, in fact, the most preferred benefit agent for use in the delivery systems of this invention. The most preferred are unsaturated ketones, especially α,β-unsaturated ketones.

The perfume ketones utilized in the benefit agent delivery systems herein can comprise any material which is chemically a ketone and which can impart a desirable odor or freshness benefit to surfaces which have been contacted with the delivery systems formed from it. The perfume ketone component can, of course, comprise more than one ketone, i.e., mixtures of ketones. Preferably, the perfume ketone is selected from buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-Ionone, Beta-Ionone, dihydro-Beta-Ionone, Gamma-Methyl so-called Ionone, Fleuramone, Dihydrojasmone, Cis-Jasmone, Iso-E-Super, Methyl-Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane, 6,7-Dihydro-1,1,2,3,3-Pentamethyl-4(5H)-Indanone, 4-Damascol, Dulcinyl or Cassione, Gelsone, Hexalon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, Tetrameran, hedione, floralozone, and mixtures thereof.

More preferably, from the above-mentioned compounds, the preferred perfume ketones are selected from Alpha Damascone, Delta Damascone, Iso Damascone, Carvone, Gamma-Methyl-Ionone, Beta-Ionone, Iso-E-Super, 2,4,4,7-Tetramethyl-oct-6-en-3-one, Benzyl Acetone, Beta Damascone, Damascenone, methyl dihydrojasmonate, methyl cedrylone, hedione, floralozone and mixtures thereof.

Perfume aldehydes useful as benefit agents herein can comprise any perfume material which is chemically an aldehyde, which can, like the perfume ketone component, also impart a desirable odor or freshness benefit to surfaces which have been contacted with the delivery systems formed from it. As with the perfume ketone benefit agents, the perfume aldehyde benefit agent component can comprise a single individual aldehyde or mixtures of two or more perfume aldehydes. In addition, the perfume aldehyde materials useful herein will preferably comprise aldehydes which are relatively “bulky.” By bulky, it is meant that the perfume aldehyde will have relatively high molecular weight and have a relatively high boiling point. For purposes of this invention, high molecular weight perfume aldehydes are those having a boiling point greater than 225° C. Further, for purposes of this invention, high molecular weight perfume aldehydes are those with a molecular weight greater than 150.

More preferably the perfume aldehydes used herein will comprise materials which have a boiling point above 250° C. and a Clog P greater than 3. Clog P is defined hereinbefore with respect to the characterization of the solubility of preferred monoamines. In an analogous manner, this same parameter can also be used to characterize preferred perfume aldehydes.

Suitable perfume aldehyde materials for use in the delivery systems herein, whether by themselves or as part of a perfume aldehyde mixture, include adoxal; anisic aldehyde; cymal; ethyl vanillin; florhydral; helional; heliotropin; hydroxycitronellal; koavone; lauric aldehyde; lyral; triplal, melonal, methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxy0hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, 1-p-menthene-q-carboxaldehyde, citral, lilial, cumin aldehyde, mandarin aldehyde, Datilat, geranial, and mixtures thereof.

More preferred perfume aldehydes are selected from citral, 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, trans-2-nonenal, lilial, trans-2-nonenal, Datilat, anisic aldehyde, geranial, 1-octanal, helional, cuminaldehyde, triplal, melonal, and mixtures thereof.

Indirect Application of Delivery Systems to Substrate Surfaces

The components of the benefit agent delivery systems herein are selected and processed such that the resulting delivery systems provide their benefit in a certain manner to substrate surfaces which have been indirectly contacted with such delivery systems. For purposes of this invention, indirect application of the delivery system occurs when a substrate surface is contacted with a dilute solution of the delivery system, such as in aqueous solution or dispersion of such a delivery system. For purposes of this invention, a “dilute” solution of the delivery system is a solution that contains a lower, i.e., less than 50%, concentration of the benefit agent when exposed to the substrate than was the concentration of the benefit agent in the delivery system prior to such exposure. For example, the benefit agent may be at one-half of the concentration it was in the delivery system in the aqueous solution or dispersion which is exposed to the substrate. Such aqueous solutions or dispersions can, of course, be formed by diluting the delivery system, or end product containing it, with water. This typically occurs when a product containing the delivery system is used for its intended purpose such as, for example, when a laundry detergent containing a perfume delivery system is used to launder fabrics. For purposes of this invention, an aqueous solution or dispersion of a delivery system is one which contains no more than 5000 ppm, preferably no more than 500 ppm, even more preferably no more than 50 ppm, and most preferably no more than 10 ppm and even sometimes no more than 1 ppm, of the benefit agent.

Indirect application of the delivery system includes any situation wherein the ultimate treatment of the substrate involved occurs with an aqueous solution or dispersion of the delivery system-containing product. This is true even if a substrate may initially be contacted with the concentrated delivery system-containing product. Thus, for example, even though a shampoo or body wash product may initially be contacted with and applied directly to hair or skin, such products are quickly diluted by the addition of water and used thereafter for indirct application of the benefit agent delivery system.

The materials used in the delivery systems are such that the system is especially effective for delivering the benefit agent to the surface of a substrate which has been indirectly contacted, i.e., via an aqueous solution or dispersion, with the product containing the delivery system. Under such conditions, the benefit agent delivered to the substrate surface will provide its benefit thereto for a longer period of time than if no amine-based compound were present in the delivery system. Of course, in determining such comparative delivery of benefit agent to a substrate surface, there must be sufficient contact of substrate with treating solution or dispersion in order to deposit at least some measurable amount of benefit agent on the surface.

Delivery System Preparation

The benefit agent delivery system suitable for use in granular forms/matrices can be prepared by simply admixing the amine-based compound and the benefit agent ketone and/or aldehyde with the matrix under conditions which are sufficient to bring about combination, e.g., thorough admixture, of these components with the liquid or granular matrix. Frequently this admixing is carried out using high shear agitation. Temperatures of from 40° C. to 65° C. may be utilized. Additional materials may also be added to the matrix in order to form the complete end product into which the delivery system is to be incorporated.

In liquid matrices, especially, on a weight basis, the ratio of amine to benefit agent can vary widely, and will frequently range from 1000:1 to 1:50. In one embodiment, the ratio of amine to benefit agent is from 1000:1 to 1:5, more typically from 100:1 to 1:2, even more typically from 50:1 to 1:1, for the two essential components. (amine compound and ketone/aldehyde benefit agent). As noted, these two components do need to be added separately, i.e., in a form such that they are unreacted with each other. Thus these two components do not have to be added to the matrix simultaneously. They are, in fact, preferably added to the matrix sequentially.

Cleaning and Fabric Treatment Products

The benefit agent delivery systems of the present invention are preferably incorporated into a wide variety of cleaning products and fabric treatment products. Such products include both laundry and cleaning compositions which are typically used for laundering fabrics and cleaning hard surfaces such as dishware, floors, bathrooms, toilet, kitchen and other surfaces in need of a prolonged or delayed release of the benefit agent. Accordingly, by laundry and cleaning compositions, these are to be understood to include not only detergent compositions which provide fabric cleaning benefits, but also compositions such as hard surface cleaning which provide hard surface cleaning benefit.

Products in which the delivery systems herein can be incorporated also include fabric treatment products such as fabric softeners or conditioners. Such products do not necessarily impart a cleaning benefit to fabrics treated therewith.

Preferred as products in which the delivery systems herein can be incorporated are those laundry and fabric treatment, e.g., softener, compositions which provide deposition of the benefit agent onto fabrics via contact of fabrics with aqueous solutions or dispersions of the products.

The effectiveness of the delivery to treated surfaces of the preferred benefit agent, perfumes, can be quantified by means of a parameter called the Dry Surface Odor Index. Such a parameter is fully described in PCT Application No. WO 00/02982. Preferably, the perfume delivery systems herein which are incorporated into cleaning and fabric treatment products will provide a Dry Surface Odor Index of more than 5 and preferably at least 10.

Cleaning products incorporating the benefit agent delivery systems of the present invention may also take the form of shampoos or body wash compositions. With such products, the substrate being cleaned is, of course, hair or skin.

In general, the benefit agent delivery systems herein can be incorporated into cleaning or fabric treatment products herein such that levels of amine plus benefit agent range from 0.005% to 10% by weight, more preferably fom 0.005% to 5%, even more preferably from 0.02% to 0.5% by weight. For cleaning products, the amine plus benefit agent combination will generally be incorporated at concentrations of from 0.005% to 10% by weight, along with from 1% to 50% by weight of a surfactant. For fabric treatment products, the amine/benefit agent combination will generally be incorporated at concentrations of from 0.005% to 5% by weight, along with from 1% to 50% by weight of a fabric softening or treating agent. The cleaning and fabric treatment products containing the delivery systems herein can comprise a wide variety of additional adjuvants which are conventional for use in products of these types. Extensive disclosure of such conventional adjuvants can be found in PCT Patent Application Nos. WO 00/02982 and WO 00/02987.

The cleaning and treatment products which contain the benefit agent delivery systems herein may take a variety of physical forms including liquids, gels or foams in aqueous or nonaquous form, granular form or tablet form. An especially preferred form for products of this type is a liquid detergent composition, e.g., a heavy duty liquid (HDL) detergent for fabric laundering.

Preparation of the benefit agent delivery systems herein and their incorporation into certain types of cleaning products can be illustrated by the following examples:

### EXAMPLE I

### Preparation of Liquid Detergent Composition

A heavy-duty liquid detergent composition in accordance with the present invention can be made as follows:

Step 1—a conventional heavy-duty liquid detergent composition is made by any conventional method known in the art;

Step 2—0.01% by weight of an amine in accordance with the present invention is added to the composition from Step 1 and the composition is then mixed for about 1-3 minutes;

Step 3—0.015% by weight of a benefit agent in accordance with the present invention is added to the amine-containing composition from Step 2 and the composition is then mixed for about 5 minutes.

Note that Step 2 and Step 3 are separate discrete addition steps.

A variety of detergent compositions are prepared having the compositions shown in the following Examples II through VI. In these examples the abbreviated component identifications have the following meanings:
LAS: Sodium linear C₁₂ alkyl benzene sulphonate
CFAA: C₁₂-C₁₄ alkyl N-methyl glucamide
HEDP: Hydroxyethane dimethylene phosphonic acid
DETPMP: Diethylene triamine penta (methylene phosphonic acid), marketed by Monsanto under the Tradename Dequest 2060
TEPAE Tetreaethylenepentaamine ethoxylate
PVP Polyvinylpyrrolidone polymer
PVNO Polyvinylpyridine-N-Oxide, with an average molecular weight of 50,000.
Brightener Disodium 4,4′-bis(2-sulphostyryl)biphenyl and/or Disodium 4,4′-bis(4-anilino-6-morpholino-1.3.5-triazin-2-yl) stilbene-2:2′-disulfonate.
Suds Suppressor-25% paraffin wax Mpt 50° C., 17% hydrophobic silica, 58% paraffin oil Granular suds suppressors 12% Silicone/silica, 18% stearyl alcohol, 70% starch in granular form
PEI Polyethyleneimine
Enzymes: Protease, amylase, cellulase and/or lipase
SRP Anionically end capped poly esters.
MEA Monoethanolamine
SCS Sodium Cumene Sulfonate
   Amine No. 1—Lupasol WF (PEI of MW 25,000)
   Amine No. 2—Lupasol G35 (PEI of MW 1200)
   Amine No. 3—N,N′-bis-(3-aminopropyl)-1,3-propanediamine
   Amine No. 4—N,N′-bis-(3-aminopropyl)-ethylenediamine
   Benefit Agent No. 1—Delta-damascone
   Benefit Agent No. 2—melanol
   Benefit Agent No. 3—triplal
   Benefit Agent No. 4—helional

### EXAMPLE II

A heavy duty liquid (HDL) detergent composition is prepared containing a benefit agent delivery system prepared as in Example I. Such a liquid detergent composition has the following formula:

| Ingredient | % by wt. |
|---|---|
| Trisodium Citrate | 3.480 |
| C12-18 Real Soap | 3.241 |
| Ethanol | 2.500 |
| MEA | 1.500 |
| Ca Formate | 0.050 |
| Propylene Glycol | 4.440 |
| Na Formate | 0.103 |
| Borax Premix (38%) | 1.500 |
| Glycerin | 2.700 |
| NaOH | 0.837 |
| Hydrophilic Dispersant | 0.650 |
| (PEI 189 E15-E18) | |
| Protease | 0.032 |
| Cellulase | 0.001 |
| Mannanase | 0.004 |
| Amylase | 0.003 |
| Suds Suppressor | 0.010 |
| DTPA | 0.150 |
| Hydrophobic Dispersant (PEI 600 E20) | 1.290 |
| Benefit Agent No. 1 according to | 0.020 |
| present invention | |
| Amine No. 1 cording to present | 0.0150 |
| invention | |
| Brightener | 0.125 |
| C12-14 Alkyl Dimethyl Amine Oxide | 0.740 |
| (Amine Oxide) | |
| C12-13 AE9 | 2.220 |
| C25AE1.1S Na Paste | 15.372 |
| NaLAS | 4.743 |
| Red HP Liquitint Dye | 0.002 |
| Additional Perfume | 0.280 |
| Water | 54.300 |

### EXAMPLE III

A heavy duty liquid (HDL) detergent composition is prepared containing a benefit agent delivery system prepared as in Example I. Such a liquid detergent composition has the following formula:

| Ingredient | % by wt. |
|---|---|
| Trisodium Citrate | 2.082 |
| C12-18 Real Soap | 0.548 |
| Ethanol | 1.340 |
| MEA | 1.150 |
| Ca Formate | 0.050 |
| Propylene Glycol | 2.500 |
| Na Formate | 1.000 |
| Glycerin | 0.350 |
| NaOH | 0.597 |
| Hydrophilic Dispersant | 0.210 |
| (PEI 189 E15-E18) | |
| Protease | 0.011 |
| Mannanase | 0.001 |
| Amylase | 0.002 |
| Suds Suppressor | 0.010 |
| Kathon | 0.001 |
| Hydrophobic Dispersant (PEI 600 E20) | 0.420 |
| Benefit Agent No. 1 according to present invention | 0.013 |
| Amine No. 2 according to present invention | 0.010 |
| Brightener | 0.040 |
| C12-13 AE9 | 1.450 |
| C25AE1.1S Na Paste | 10.173 |
| NaLAS | 3.098 |
| Liquitint Blue 65 | 0.016 |
| Additional Perfume | 0.260 |
| Water | 74.867 |

### EXAMPLE IV

### Liquid Detergent Composition

A heavy duty liquid (HDL) detergent composition is prepared containing a benefit agent delivery system prepared as in Example I. Such a liquid detergent composition has the following formula:

| Component | Wt. % |
|---|---|
| C₁₂₋₁₅ alkyl ether (2.5) sulfate | 19.0 |
| C₁₂₋₁₃ alkyl ethoxylate (9.0) | 2.00 |
| C₁₂₋₁₄ glucose amide | 3.50 |
| Citric Acid | 3.00 |
| C₁₂₋₁₄ Fatty Acid | 2.00 |
| MEA | to pH 8 |
| Ethanol | 3.41 |
| Propanediol | 6.51 |
| Borax | 2.5 |
| PEI - Lupasol G (MW-100) | 0.00075 |
| Damascone | 0.01 |
| Dispersant | 1.18 |
| Na Toluene Sulfonate | 2.50 |
| Dye, Brighteners, Enzymes, Preservatives, Suds | Balance |
| Suppressor, Other Minors, Water | 100% |

### EXAMPLE V

### Liquid Detergent Composition

The following liquid detergent formulations are prepared according to the present invention:

| | A | B | C | D | E |
|---|---|---|---|---|---|
| LAS | 11.5 | 9.0 | — | 4.0 | — |
| C25E2.5S | — | 3.0 | 18.0 | — | 16.0 |
| C45E2.25S | 11.5 | 3.0 | — | 16.0 | — |
| C23E9 | — | 3.0 | 2.0 | 2.0 | 1.0 |
| C23E7 | 3.2 | — | — | — | — |
| CFAA | — | — | 5.0 | — | 3.0 |
| TopPalmKernel Fatty | 2.0 | — | 2.0 | 0.5 | 2.0 |
| Acid | | | | | |
| Citric (50%) | 6.5 | 1.0 | 2.5 | 4.0 | 2.5 |
| Ca and/or Ca formate | 0.6 | 0.7 | 0.2 | 0.05 | 0.05 |
| SCS | 4.0 | 1.0 | 3.0 | 1.2 | — |
| Borate | 0.6 | — | 3.0 | 2.0 | 3.0 |
| Na hydroxide | 6.0 | 2.0 | 3.5 | 4.0 | 3.0 |
| Ethanol | 2.0 | 1.0 | 4.0 | 4.0 | 3.0 |
| 1,2 Propanediol | 3.0 | 2.0 | 8.0 | 8.0 | 5.0 |
| Monoethanolamine | 3.0 | 1.5 | 1.0 | 2.5 | 1.0 |
| TEPAE | 2.0 | — | 1.0 | 1.0 | 1.0 |
| Enzymes | 0.03 | 0.01 | 0.03 | 0.02 | 0.02 |
| Amine No. 3 | 0.015 | 0.0075 | 0.00375 | 0.2 | 0.045 |
| according to present | | | | | |
| invention | | | | | |
| Benefit Agent No. 2 | 0.02 | 0.01 | 0.005 | 0.015 | 0.0075 |
| according to present | | | | | |
| invention | | | | | |
| SRP | 0.2 | — | 0.1 | — | — |
| DTPA | — | — | 0.3 | — | — |
| PVNO | — | — | 0.3 | — | 0.2 |
| Brightener | 0.2 | 0.07 | 0.1 | — | — |
| Suds suppressor | 0.04 | 0.02 | 0.1 | 0.1 | 0.1 |

| | |
|---|---|
| Miscellaneous | Balance to 100% |
| and water | |

### EXAMPLE VI

### Liquid Detergent Composition

Heavy duty liquid fabric cleaning compositions in accordance with the invention are prepared as follows:

| | A | B |
|---|---|---|
| LAS acid form | — | 25.0 |
| Citric acid | 5.0 | 2.0 |
| 25AS acid form | 8.0 | — |
| 25AE2S acid form | 3.0 | — |
| 25AE7 | 8.0 | — |
| CFAA | 5 | — |
| DETPMP | 1.0 | 1.0 |
| PEI - Lupasol PR8515 (MW-2000) | 0.06 | 0.1 |
| Damascone | 0.02 | 0.015 |
| Lilial | 0.06 | 0.05 |
| Fatty acid | 8 | — |
| Oleic acid | — | 1.0 |
| Ethanol | 4.0 | 6.0 |
| Propanediol | 2.0 | 6.0 |
| Coco-alkyl dimethyl | — | 3.0 |
| hydroxy ethyl ammonium | | |
| chloride | | |
| Smectite clay | — | 5.0 |
| PVP | 2.0 | — |

| | |
|---|---|
| Water/Minors | Up to 100% |

### EXAMPLE VII

### Liquid Detergent Composition

Heavy-duty liquid fabric cleaning compositions in accordance with the invention are prepared as follows:

| | A | B | C |
|---|---|---|---|
| C25AES | 18.0 | 15.0 | 14.0 |
| LAS | 5.8 | 5.0 | 4.0 |
| C₈₋₁₀ Amine | 1.4 | 2.0 | — |
| Nonionic 24-7 | 2.8 | 2.0 | 3.0 |
| Citric acid | 2.5 | 3.0 | 3.0 |
| Fatty acid | 8.5 | 3.0 | 3.0 |
| Enzymes | 0.02 | 0.02 | 0.006 |
| Boric acid | 2.0 | 2.0 | 2.0 |
| Ethoxylate tetraethylene pentaimine | 0.9 | 1.0 | 1.0 |
| Polyethylene imine ethoxylated | 0.7 | — | 1.0 |
| DETPMP | 0.3 | — | — |
| PEI - Lupasol P (MW-750,000) | 0.04 | 0.1 | 0.044 |
| Damascone | 0.02 | 0.02 | — |
| Lilial | 0.02 | 0.02 | — |
| Hexyl Cinnamic Aldehyde | — | 0.01 | 0.02 |
| Florhydral | — | — | 0.05 |
| HEDP | 0.35 | — | — |
| Ethanol | 1.0 | 3.0 | 3.0 |
| 1,2,propanediol | 8.0 | 4.0 | 5.0 |
| MEA | 9.8 | 2.0 | 2.0 |
| Na Cumene Sulfonate | 2.0 | — | — |
| Suds suppressors | 0.25 | 0.01 | 0.01 |

| | |
|---|---|
| Minors (perfumes, brighteners) | Up to 100% |
| and water | |

### EXAMPLE VIII

### Granular Detergent Composition

A heavy duty granular detergent (HDG) composition is prepared containing the pro-perfume composition of Example I. Such a granular detergent composition has the following formula:

| Component | Wt. % |
|---|---|
| C₁₂ Linear alkyl benzene sulfonate | 9.31 |
| C₁₄₋₁₅ alkyl sulfonate | 12.74 |
| Zeolite Builder | 27.79 |
| Sodium Carbonate | 27.31 |
| PEG 4000 | 1.60 |
| Dispersant | 2.26 |
| C₁₂₋₁₃ alkyl ethoxylate (E9) | 1.5 |
| Sodium Perborate | 1.03 |
| Soil Release Polymer | 0.41 |
| PEI - Lupasol SK (MW-2,000,000) | 0.04 |
| Damascone | 0.02 |
| Lilial | 0.03 |
| Florhydral | 0.01 |
| Enzymes | 0.59 |
| Brightener, Suds Suppressor, Other Minors, Moisture | 0.3 |
| Sulfate | Balance up to |
| | 100% |

### EXAMPLE IX

Body Wash

| Ingredient | A | A | A |
|---|---|---|---|
| Sodium Laureth Sulfate | 7.5 | 8.5 | 8.2 |
| Cocamidopropyl Betaine | 6.5 | 5.5 | 4.5 |
| Sodium Lauroyl Sarcosinate | 0.75 | 0.65 | 1.2 |
| Citric Acid | 0.26 | 0.33 | 0.38 |
| Guar | 0.50 | 0.30 | 0.30 |
| Hydroxypropyltrimonium | | | |
| Chloride | | | |
| Lauryl Alcohol | 0.65 | 0.80 | 0.77 |
| DMDM Hydantoin | 0.21 | 0.26 | 0.11 |
| Sodium Benzoate | 0.25 | 0.15 | 0.18 |
| Disodium EDTA | 0.10 | 0.05 | 0.20 |
| Amine No. 3 according to | 1.8 | 0.8 | 0.35 |
| present invention | | | |
| Amine No. 4 according to | — | — | 0.15 |
| present invention | | | |
| Benefit Agent No. 3 | 0.7 | 2.1 | 1.1 |
| according to present | | | |
| invention | | | |
| Water/Carriers/aesthetics | balance | balance | balance |

### EXAMPLE X

Shampoo

| | | | |
|---|---|---|---|
| Ammonium Laureth/Lauryl | 16 | 14 | 20 |
| Sulfate | | | |
| Glycol Distearate | 1.5 | 1.1 | 1.6 |
| Dimethicone | 1.4 | 1.1 | 1.8 |
| Cetyl Alcohol | 0.90 | 1.2 | 1.4 |
| Cocamide MEA | 0.75 | 0.95 | 0.55 |
| Sodium Chloride | 0.65 | 1.0 | 1.3 |
| Polyquaternium-10 (LR-400) | 0.50 | 0.30 | 0.20 |
| Sodium Citrate | 0.60 | 0.40 | 0.50 |
| Hydrogenated Polydecene | 0.30 | 0.20 | 0.70 |
| Sodium Benzoate | 0.20 | 0.35 | 0.40 |
| Disodium EDTA | 0.12 | 0.085 | 0.15 |
| Trimethylolpropane | 0.10 | 0.15 | 0.10 |
| Tricaprylate/Tricaprate | | | |
| Citric Acid | 0.040 | 0.050 | 0.040 |
| Pro vitamins | 0.060 | — | 0.030 |
| Methylchloroisothiazolinone/ | 0.00038 | 0.0010 | 0.00031 |
| Methylisothiazolinone | 0.00012 | 0.00018 | 0.00028 |
| Amine according to present | 1.0 | 0.65 | 0.10 |
| invention | | | |
| Benefit Agent according to present | 0.50 | 0.75 | 1.2 |
| invention | | | |
| Water/Carriers/Aesthetics | Balance | balance | balance |

1. A process comprising separately adding:
a.) an amine-based compound having a molecular weight of at least 100 Daltons, at least 10% of the amino groups of said amine-based compound being primary amino groups; and
b.) a benefit agent in the form of an aldehyde or ketone to a granular or liquid matrix.

2. A process according to claim 1 wherein the amine-based compound is a polyamine having a molecular weight of at least 150 Daltons and having from 15% to 80% of its amino groups as primary amino groups.

3. A process according to claim 1 wherein said amine-based compound has an Odor Intensity Index of less than that of a 1% solution of methylanthranilate in dipropylene glycol.

4. A process according to claim 1 wherein the benefit agent is selected from perfumes, flavors, pharmaceuticals and biocontrol agents.

5. A process according to claim 4 wherein the benefit agent comprises an aldehyde moiety and/or a ketone moiety.

6. A process according to claim 1 wherein said amine-based compound is a non-aromatic amine.

7. A process according to claim 4 wherein the benefit agent is a perfume compound selected from Alpha Damascone, Delta Damascone, Iso Damascone, Carvone, dihydro-Beta-Ionone, Beta-Ionone, Gamma-Methyl-Ionone, Isocyclemone E, 2,4,4,7-Tetramethyl-oct-6-en-3-one, Benzyl Acetone, Beta Damascone, Damascenone, methyl dihydrojasmonate, methyl cedrylone, hedione, floralozone, citral, 1-decanal, benzaldehyde, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, trans-2-nonenal, lilial, Datilat, anisic aldehyde, geranial, 1-octanal, cuminaldehyde, 2,6-dimethyl-5-Heptenal and mixtures thereof.

8. A process according to claim 4 wherein the amine-based compound is selected from polyethyleneimine polymers; partially alkoxylated polyethylene polymers, polyethyleneimine polymers with hydroxyl groups, diaminobutane dendrimers, polylysine, cross-linked polylysine, N,N′-bis-(3-aminopropyl)-1,3-propanediamine linear or branched; N,N′-bis-(3-aminopropyl)ethylenediamine; 1,4-bis-(3-aminopropyl) piperazine, 1,5-pentanediamine, 1,6-hexanediamine, 1,3 pentanediamine, 3-dimethylpropanediamine, 1,2-cyclohexanediamine, 1,3-bis(aminomethyl)cyclohexane, tripropylenetetraamine, bis(3-aminopropyl)piperazine, dipropylenetriamine, tris(2-aminoethylamine), tetraethylenepentamine, bishexamethylenetriamine, bis(3-aminopropyl) 1,6-hexamethylenediamine, 3,3′-diamino-N-methyldipropylamine, 2-methyl-1,5-pentanediamine, N,N,N′,N′-tetra(2-aminoethyl)ethylenediamine, N,N,N′,N′-tetra(3-aminopropyl)-1,4-butanediamine, pentaethylhexamine, 1,3-diamino-2-propyl-tert-butylether, isophorondiamine, 4,4′,-diaminodicyclohylmethane, C₁₂-C₁₄ Sternamines, C₁₂-C₁₄ Sternamine(propyleneamine)n with n=¾ and mixtures thereof.

9. A process according to claim 1 wherein the amine-based compound comprises a monoamine.

10. A process according to claim 9 wherein the monoamine comprises a hydroxy and/or alkoxy functional group.

11. A process according to claim 10 wherein said monoamine has a ClogP greater than 1.

12. A process according to claim 11 which contains a primary monoamine or a combination of primary and secondary monoamines.

13. A process comprising separately adding, in a weight ratio of amine compound to perfume ranging from 1000:1 to 1:50:
a.) an amine-based compound having a molecular weight of at least 150 Daltons, at least 10% of the amino groups of said amine-based compound being primary amino groups; and
b.) a perfume selected from Damascone, alpha-Damascone, beta-Damascone, delta-Damascone, iso-Damascone, beta-Ionone, lilial, alpha-n-hexylcinnamic aldehyde, alpha-n-amylcinnamic aldehyde, cymal, lyral butylcinnamic aldehyde, datilat, 2,4-dimethyl-3-cyclohexene-1-carboxaldehyde, 2,6-dimethyl-5-Heptenal and mixtures thereof, to a liquid matrix.

14. A process according to claim 13 wherein said amine-based compound is selected from Sternamines C12-C14, Sternamines C12-C14(propyleneamine)n with n=¾, N,N′-bis-(3-aminopropyl)-1,3-propanediamine linear or branched; N,N′-bis-(3-aminopropyl) ethylenediamine, and mixtures thereof.

15. A cleaning or fabric treatment product containing from 0.005% to 10% by weight of a benefit agent or perfume delivery system produced according to the process of claim 14.

16. A cleaning composition comprising from 1% to 50% by weight of a surfactant and from about 0.005% to 10% by weight of a perfume delivery system produced according to the process of claim 14.

17. A cleaning composition according to claim 16 which is in the form of a liquid detergent composition.

18. A cleaning composition according to claim 16 which is in the form of a shampoo or body wash.

19. A fabric treatment composition comprising from 1% to 50% by weight of a fabric softening or treatment agent and from 0.005% to 5% of a benefit agent or perfume delivery system produced according to the process of claim 14.
